SEQUENCE LISTING (32 sequences)

SEQ ID NO: 1 - protein, 121 aa, synthetic construct
QVQLQQSGAELARPGASVKLSCKASGYTFTSYGLSWVKQRTGQGLEWIGEIFPRSGNSYYNEKFKGKAALTADKSSSTAYMQLSSLTSEDSAVYFCARHYYSSLYYAMDYWGQGTSVTVSS

SEQ ID NO: 2 - protein, 107 aa, synthetic construct
DIQVTQSPASLSASVGESVSITCGTNENLYGALNWYQRKQGKSPQLLIYGATNLADGMSSRFSGSGSGRQYSLKISSLHPDDVATYYCQNVLSTPRTFGGGTKLEIK

SEQ ID NO: 3 - protein, 117 aa, synthetic construct
EVQLVESGGGLVKPGGSLKLSCAASGFSFSDYYMYWVRQAPEKRLEWVASISFDGTYTYYTDRVKGRFTISRDNAKNNLYLQMSSLKSEDTAMYYCARDRPAWFPYWGQGTLVTVSA

SEQ ID NO: 4 - protein, 112 aa, synthetic construct
DVLMTQIPLSLTVSLGDQASISCRSSQSIVHSNGNTYLEWYLQRPGQSPKLLIYKVSNRFSGVPDRFSGSGSGTDFTLKISRVEAEDLGVYYCFQGSHVPLTFGAGTKLELK

SEQ ID NO: 5 - protein, 119 aa, synthetic construct
EVKLQESGPELVKPGASVTISCKASGYTFTDYYINWVKQRPGQGLEWIGWLFPGSGTTYYNEKFKGKATLTVAKSSSTAYMLLSSLTSEDSAVYFCARWGFGNYAFAYWGQGTLVTVSA

SEQ ID NO: 6 - protein, 107 aa, synthetic construct
DIVLTQSQKFMSATVGDRVSITCKASQNVGTAVAWYQQKPGQSPKLLIYSPSSRNTGVPDRFTGSGSGTDFTLTISSVQSEDLADYFCQQYSTYPRTFGGGTKLEIK

SEQ ID NO: 7 - protein, 116 aa, synthetic construct
EVQLVESGGGLVQPGGSLRLSCAASGFTFSSYAMSWVRQTPGKGLEWVAVIDSNGGSTYYPDTVKDRFTISRDNSKNTLYLQMNSLRAEDTAVYYCSSYTNLGAYWGQGTLVTVSA

SEQ ID NO: 8 - protein, 107 aa, synthetic construct
DIQMTQSPSSLSASVGDRVTITCRASQDISGYLNWLQQKPGGAIKRLIYTTSTLDSGVPKRFSGSGSGTDFTLTISSLQSEDFATYYCLQYASSPFTFGGGTKVEIK

SEQ ID NO: 9 - protein, 8 aa, synthetic construct
GYTFTSYG

SEQ ID NO: 10 - protein, 8 aa, synthetic construct
IFPRSGNS

SEQ ID NO: 11 - protein, 14 aa, synthetic construct
ARHYYSSLYYAMDY

SEQ ID NO: 12 - protein, 6 aa, synthetic construct
ENLYGA

SEQ ID NO: 13 - skipped

SEQ ID NO: 14 - protein, 9 aa, synthetic construct
QNVLSTPRT

SEQ ID NO: 15 - protein, 5 aa, synthetic construct
DYYMY

SEQ ID NO: 16 - protein, 17 aa, synthetic construct
SISFDGTYTYYTDRVKG

SEQ ID NO: 17 - protein, 8 aa, synthetic construct
DRPAWFPY

SEQ ID NO: 18 - protein, 16 aa, synthetic construct
RSSQSIVHSNGNTYLE

SEQ ID NO: 19 - protein, 7 aa, synthetic construct
KVSNRFS

SEQ ID NO: 20 - protein, 9 aa, synthetic construct
FQGSHVPLT

SEQ ID NO: 21 - protein, 5 aa, synthetic construct
DYYIN

SEQ ID NO: 22 - protein, 17 aa, synthetic construct
WLFPGSGTTYYNEKFKG

SEQ ID NO: 23 - protein, 10 aa, synthetic construct
WGFGNYAFAY

SEQ ID NO: 24 - protein, 11 aa, synthetic construct
KASQNVGTAVA

SEQ ID NO: 25 - protein, 7 aa, synthetic construct
SPSSRNT

SEQ ID NO: 26 - protein, 9 aa, synthetic construct
QQYSTYPRT

SEQ ID NO: 27 - protein, 8 aa, synthetic construct
GFTFSSYA

SEQ ID NO: 28 - protein, 8 aa, synthetic construct
IDSNGGST

SEQ ID NO: 29 - protein, 9 aa, synthetic construct
SSYTNLGAY

SEQ ID NO: 30 - protein, 6 aa, synthetic construct
QDISGY

SEQ ID NO: 31 - skipped

SEQ ID NO: 32 - protein, 9 aa, synthetic construct
LQYASSPFT

(19) United States
(12) Patent Application Publication
(10) Pub. No.: US 2025/0035633 A1
(43) Pub. Date: Jan. 30, 2025

(54) COMPOSITIONS AND METHODS FOR DETECTING CADHERIN-17 PROTEIN

(71) Applicant: ARBELE Limited, Shatin, N.T. (HK)

(72) Inventors: John Moonching LUK, Bellevue, WA (US); Deepak Narayanan IYER, Tung Chung (HK)

(21) Appl. No.: 18/696,902

(22) PCT Filed: Oct. 8, 2022
(86) PCT No.: PCT/US2022/077826

§ 371 Date: Mar. 28, 2024

Related U.S. Application Data
(60) Provisional application No. 63/253,609, filed on Oct. 8, 2021.

(51) Int. Cl. G01N 33/574; C07K 16/28; C12Q 1/28; G01N 33/58; G01N 33/68

(52) U.S. Cl. CPC G01N 33/57446; C07K 16/2896; C12Q 1/28; G01N 33/582; G01N 33/6893; C07K 2317/565; G01N 2333/70596; G01N 2333/908

(57) ABSTRACT

A method for screening a subject for cancer by determining the amount of CDH17 protein in a sample from the subject, the method comprising the steps of contacting the sample to a capture antibody having a binding affinity to CDH17, wherein any CDH17 protein in the sample is configured to bind to the capture antibody to provide a bound sample, contacting the bound sample to a detection molecule to provide a detection sample, wherein the detection molecule comprises a sensing signal molecule conjugated to a secondary antibody having a binding affinity to the CDH17 protein, generating a sensing signal through the sensing signal molecule bound to the detection sample, determining the amount of the sensing signal, and determining the amount of the CDH17 protein in the sample based on the amount of the sensing signal.

### CROSS REFERENCE TO RELATED APPLICATIONS

This application claims the benefit of the filing date of U.S. Provisional Application Ser. No. 63/253,609 filed Oct. 8, 2021, under 35 U.S.C. 119 (e), the entire disclosures of which are incorporated by reference herein.

### TECHNICAL FIELD

The present application relates to methods useful for the detection of cancer. In particular, the present application relates to detecting CDH17 protein in a human biofluid sample.

### BACKGROUND

Unless otherwise indicated herein, the materials described in this section are not prior art to the claims in this application and are not admitted to be prior art by inclusion in this section.

Gastrointestinal (GI, i.e., stomach, liver, esophagus, pancreas, and colorectal) cancers are leading causes of morbidity and mortality worldwide. As per the International Agency for Research on Cancer, GI cancers account for 4.8 million cancer cases and 1 in 3 cancer related mortalities worldwide, with colorectal cancer being the leading player (Sung et al, 2021). The five-year survival rate for patients diagnosed with colorectal cancer is 91% for localized disease, 72% for regional metastases, and 14% for stage IV disease (Gonzalez-Pons, 2015). Early detection of the disease can enable curative surgery for most tumors (Siegel 2017). Specifically, identification of non-malignant lesions, adenomas, and nodules within the gastrointestinal canal that are recognized high risk factors for the development of a malignant disease allow for a significant improvement in the overall disease outcome. Currently applicable blood based clinical biomarkers such as CEA and CA19-9 lack the sensitivity and specificity required for early detection of GI cancers (Lech 2016).

Cadherin-17 (CDH17) is a biomarker for GI cancers characterized by its overexpression in stomach, liver, and colorectal cancers but not in normal tissues from healthy adults. CDH17 is also a useful immunohistochemical marker for diagnosis of adenocarcinomas of the digestive system (Su et al 2008). Moreover, CDH17 is highly expressed in metastatic cancers, and the blockage of CDH17 expression and functions can markedly reduce lung metastasis of hepatocellular carcinoma (HCC) (Lee et al 2010). While CDH17 functions as a valid GI cancer diagnostic biomarker, an improvement in the detection strategy of CDH17 is essential to apply it for large scale screening approaches at a higher test sensitivity and specificity.

### SUMMARY

The following summary is illustrative only and is not intended to be in any way limiting. In addition to the illustrative aspects, embodiments, and features described above, further aspects, embodiments, and features will become apparent by reference to the drawings and the following detailed description.

In one aspect, the application provides methods for detecting or screening a subject for cancer. The method includes the steps of determining the amount of CDH17 protein in a sample from the subject. The subject may carry cancer or may be carrying a pre-cancerous condition. The sample may or may not contain CDH17 protein. The method is used to detect the presence of CDH17 protein, if any, in the sample, therefore, characterizing the cancer or pre-cancerous condition that the subject may be suffer from.

In one embodiment, the method includes the steps of contacting the sample to a capture antibody having a binding affinity to CDH17, wherein any CDH17 protein in the sample is configured to bind to the capture antibody to provide a bound sample, contacting the bound sample to a detection molecule to provide a detection sample, wherein the detection molecule comprises a sensing signal molecule conjugated to a secondary antibody having a binding affinity to the CDH17 protein, generating a sensing signal through the sensing signal molecule bound to the detection sample, determining the amount of the sensing signal, and determining the amount of the CDH17 protein in the sample based on the amount of the sensing signal.

In one embodiment, the method may further include the step of determining the probability of the subject carrying CDH17 positive precancerous condition based on the amount of CDH17 protein in the sample. In one embodiment, the method may further include the step of determining the probability of the subject carrying the CDH17 positive cancer based on the amount of the CDH17 protein in the sample.

In one embodiment, the sensing signal molecule comprises a peroxidase enzyme and the sensing signal comprises an oxidized substrate. The peroxidase enzyme oxidizes a substrate to provide the oxidized substrate.

In one embodiment, the sensing signal molecule comprises a fluorescent labeling reagent and the sensing signal comprises fluorescence signal. In one embodiment, the fluorescent labeling reagent comprises europium.

In one embodiment, the method include contacting a sample potentially containing the CDH17 protein to a capture antibody having a binding affinity for the CDH17 protein, allowing the CDH17 protein to bind to the capture antibody to provide a bound sample, contacting a detection molecule to the bound sample, wherein the detection molecule comprises a peroxidase enzyme conjugated to a secondary antibody having an affinity to the CDH17 protein, oxidizing a substrate with the peroxidase enzyme to provide an oxidized substrate, determining the amount of the oxidized substrate, and determining the amount of the CDH17 protein based on the amount of the oxidized substrate.

In one embodiment, the method includes the steps of contacting a sample potentially containing the CDH17 protein to a capture antibody having a binding affinity for the CDH17 protein, allowing the CDH17 protein to bind to the capture antibody to provide a bound sample, contacting a detection molecule to bind to the bound sample, wherein the detection molecule comprises a fluorescent labeling agent (such as europium) conjugated to a secondary antibody having an affinity to the CDH17 protein, determining the amount of the fluorescence signal produced, and determining the amount of the CDH17 protein based on the fluorescence signal produced.

In one embodiment, the capture antibody comprises an amino acid sequence having at least 75%, 80%, 85%, 90%, 95%, 98%, or 99% sequence identity to SEQ ID NO: 1, 2, 3, 4, 5, or 6. In one embodiment, the capture antibody comprises 3 heavy chain complimentary determining regions (CDRs) having the SEQ ID NO: 9, 10, 11 and 3 light chain CDRs having the SEC ID NO: 12, 13, 14. In one embodiment, the capture antibody comprises 3 heavy chain CDRs having the SEQ ID NO: 15, 16, 17 and 3 light chain CDRs having the SEQ ID NO: 18, 19, 20. In one embodiment, the capture antibody comprises 3 heavy chain CDRs having the SEQ ID NO: 21, 22, 23 and 3 light chain CDRs having the SEQ ID NO: 24, 25, 26.

In one embodiment, the secondary antibody comprises an amino acid sequence having at least 75%, 80%, 85%, 90%, 95%, 98%, or 99% sequence identity to SEQ ID NO: 7 or 8. In one embodiment, the secondary antibody comprises 3 heavy chain CDRs having the SEQ ID NO: 27, 28, 29 and 3 light chain CDRs having the SEC ID NO: 30, 31, 32.

In one embodiment, the capture antibody may be carried on a biosensor. In one embodiment, the capture antibody may be immobilized on a solid surface. In one embodiment, the capture antibody may be immobilized on a plate.

The cancer that can be detected with the disclosed methods may be a CDH17 positive cancer. In one embodiment, the cancer may be a gastrointestinal cancer.

The sample may be a bodily fluid from the subject including, for example, peripheral blood, serum, plasma, urine, saliva, bone marrow, pleural or peritoneal fluid, or intestinal fluid. In one embodiment, the subject may be a human.

In a further aspect, the application may include a monoclonal antibody having an affinity to CDH17. In one embodiment, the antibody may comprise an amino acid sequence having at least 75%, 80%, 85%, 90%, 95%, 98%, 99% sequence identity to SEQ ID NO 1, 2, 3, 4, 5, or 6. In one embodiment, the antibody may comprise an amino acid sequence having at least 75%, 80%, 85%, 90%, 95%, 98%, 99% sequence identity to SEQ ID NO 7 or 8.

In one embodiment, the antibody may comprise 3 heavy chain CDRs having the SEQ ID NO: 9, 10, 11 and 3 light chain CDRs having the SEC ID NO: 12, 13, 14. In one embodiment, the antibody may comprise 3 heavy chain CDRs having the SEQ ID NO: 15, 16, 17 and 3 light chain CDRs having the SEC ID NO: 18, 19, 20. In one embodiment, the antibody may comprise 3 heavy chain CDRs having the SEQ ID NO: 21, 22, 23 and 3 light chain CDRs having the SEC ID NO: 24, 25, 26. In one embodiment, the antibody may comprise 3 heavy chain CDRs having the SEQ ID NO: 27, 28, 29 and 3 light chain CDRs having the SEC ID NO: 30, 31, 32.

### BRIEF DESCRIPTION OF THE DRAWINGS

The foregoing and other features of this disclosure will become more fully apparent from the following description and appended claims, taken in conjunction with the accompanying drawings. Understanding that these drawings depict only several embodiments arranged in accordance with the disclosure and are, therefore, not to be considered limiting of its scope, the disclosure will be described with additional specificity and detail through use of the accompanying drawings, in which:

FIG. 1 shows (a) the expression of CDH17 protein in multiple GI cancer tissues estimated using IHC assay, and (b) distinctive expression pattern of CDH17 protein in non-tumor (NT) colon and CRC tissues;

FIG. 2 shows (a) the high correlation of CDH17 protein expression within FFPE tissues (T/N) (determined by immunohistochemistry) and the Plasma (CRC) (determined by ELISA) specimens of the patients, and (b) the high correlation of CDH17 protein expression within serum samples and plasma samples within the same patients as determined by ELISA;

FIG. 3 shows (a) the measurement of CDH17 protein concentration (using BLI assay) in non-cancerous individuals, as well as patients with colorectal adenoma or colorectal cancer, and (b) clinical correlation of high CDH17 expression with an increased distant metastasis;

FIG. 4 shows (a) the measurement of CDH17 protein concentration (using ELISA) in non-cancerous individuals, adenoma, and patients at increasing stages of colorectal cancer, (b) the measurement of CDH17 protein concentration (using TRFIA) in non-cancerous individuals, patients at early or late stages of colorectal cancer, and (c) clinical correlation of high CDH17 expression with an increased (c1) tumor invasion, (c2) nodal metastasis, and (c3) distant metastasis;

FIG. 5 shows the (a) schematic representation of the three assay platforms (A) CDH17-BLI assay, (B) CDH17-ELISA, and (C) CDH17-TRFIA that enable quantitation of CDH17 protein in liquid samples, and (b) the standardization and sensitivity of the (b1) CDH17-BLI assay, (b2) CDH17-ELISA, and (b3) CDH17-TRFIA for quantifying captured CDH17. The standard curve is established using recombinant CDH17-hFc at multiple concentrations and extends down to pg/mL; and

FIG. 6 shows the (a) Coomassie blue staining of anti-CDH17 antibodies Lic3, 9A6, and 7C5 detecting CDH17, (b) CD17 domain binding specificity determination for Lic3 (domain 2), 9A6 (domain 4), and 7C5 (domain 1), to CDH17-hFc protein, and (c) binding affinity analysis of (c1) Lic3, (c2) 9A6, and (c3) 7C5 to CDH17-hFc protein as determined by OCTET (top) and ELISA (bottom).

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented herein. It will be readily understood that the aspects of the present disclosure, as generally described herein, and illustrated in the Figures, can be arranged, substituted, combined, separated, and designed in a wide variety of different configurations, all of which are explicitly contemplated herein.

The application is generally drawn, inter alia, to compositions, methods, apparatus, systems, devices, and/or computer program products related to cancer diagnosis.

In one embodiment, the application relates to a method for detecting CDH17 protein in a human biofluid sample. CDH17, also known as liver-intestine cadherin, belongs to the 7D-cadherin superfamily that functions as a peptide transporter and a cell adhesion molecule to help maintain tissue structural integrity in the epithelia. The protein is commonly expressed in fetal liver and gastrointestinal tract during embryogenesis but is silenced in adult liver and gastric tissues (Lee et al, 2010). Although, CDH17 shows a high expression in several gastrointestinal cancers including, gastric cancer, hepatocellular carcinoma, colorectal cancer, pancreatic cancer, esophageal carcinoma (Liu et al 2019, Qiu et al 2013, Bartolome et al 2014, Panarelli et al 2012). Moreover, CDH17 expression directly correlates with the disease stage as well as the presence of distant metastasis (Park et al 2011, Bartolome et al 2014, Takamura et al 2004). As a marker of early disease detection, CDH17 expression levels have also been reported to be increased in precancerous tissues such as gastric intestinal metaplasia and spasmolytic polypeptide-expressing metaplasia. (Lee H J et al 2010). Therefore, CDH17 has a high clinical utility as a diagnostic biomarker for the early detection for precancerous conditions as well as developing malignancies across multiple gastrointestinal cancers (see PCT/US2019/032752). Although there is a clear lack of robust and accurate assays for a rapid and an accurate detection of CDH17 in liquid biopsies. Consequently, using a highly sensitive platform to detect Cadherin-17 in the clinic will significantly improve early detection of cancer thereby improving disease outcomes drastically. In this regard, this disclosure provides, among others, compositions, reagents, and methods for accurately quantifying CDH17 expression in blood samples (liquid biopsy) with an extremely high diagnostic sensitivity and specificity.

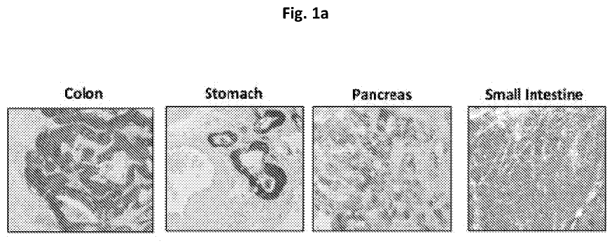

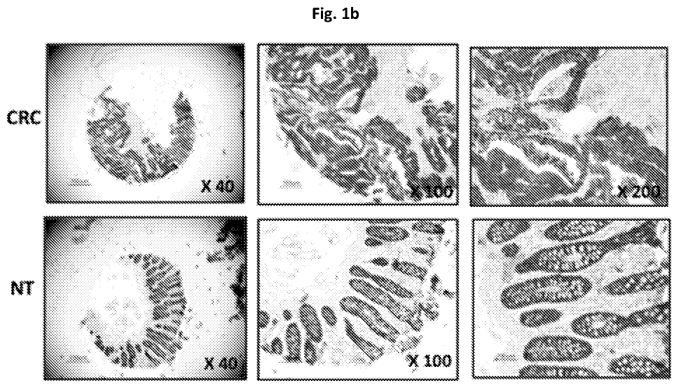

The expression of CDH17 in the tissues can be determined using immunohistochemistry (IHC). Majority of GI tissues express CDH17 (FIG. 1a). However, a significant increase in the expression of CDH17 is observed between the normal tissue and the cancerous GI tissue, with a representative example of colorectal cancer shown in FIG. 1b.

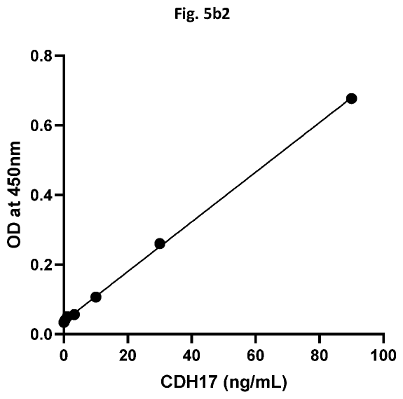

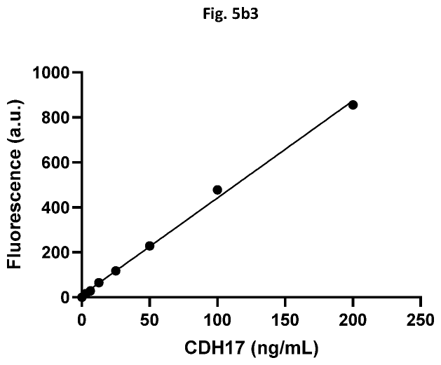

In one embodiment, the disclosure relates to the development of a highly sensitive and specific assay for the accurate and rapid quantification of CDH17 in blood specimens. Three platforms, CDH17-BLI assay, CDH17-ELISA, and CDH17-TRFIA as shown in FIG. 5a, were compared to develop assays with the highest sensitivity and dynamic range for the detection of CDH17 in biofluids. Of these, the CDH17-BLI assay has the advantage of being the quickest assay involving limited number of steps. The Bio-Layer Interferometry (BLI) is a label-free assay for measuring biomolecular interactions. For protein quantitation, the system utilizes optic fiber biosensors precoated with an immobilized protein. The binding between a ligand immobilized on the biosensor tip surface and an analyte in solution produces an increase in optical thickness at the biosensor tip, which results in a wavelength shift, which is a direct measure of the change in thickness of the biological layer. Such shifts in the interference pattern are measured in real time, thus allowing the system to monitor protein binding specificity, and concentration with high precision and accuracy. Owing to these mechanics, the BLI technology does not suffer from the interference of the sample matrix (cell culture supernatant, liquid biopsies like urine, blood, etc.) on the sensitive quantitation of the target metabolite. Moreover, undiluted samples may be used directly for metabolite quantification using the BLI assay. In comparison to BLI assay, ELISA and TRFIA both depend on immobilizing the capture antibody on a hydrophilic microplate surface following which it is exposed to the antigen (CDH17) within the sample. While ELISA depends on a peroxidase-led absorbance-based detection technology, TRFIA incorporates heavy metals such as europium which offer a large stokes shift and consequently a greater fluorescence signal retention for a longer time.

The major steps of analytical validation include:

(A) Identification of effective capture and detection anti-CDH17 antibodies.

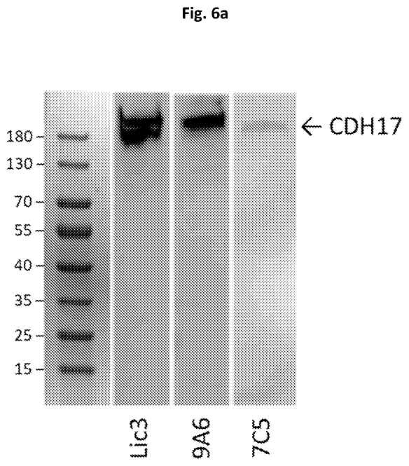

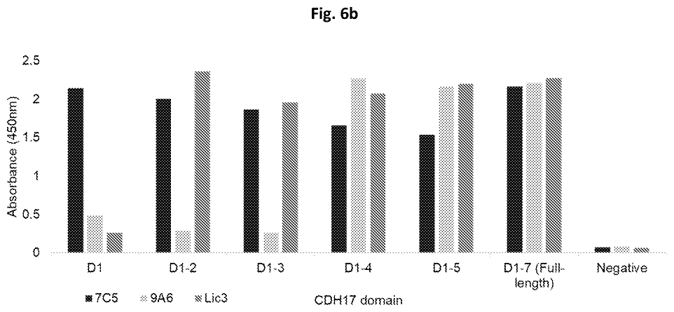

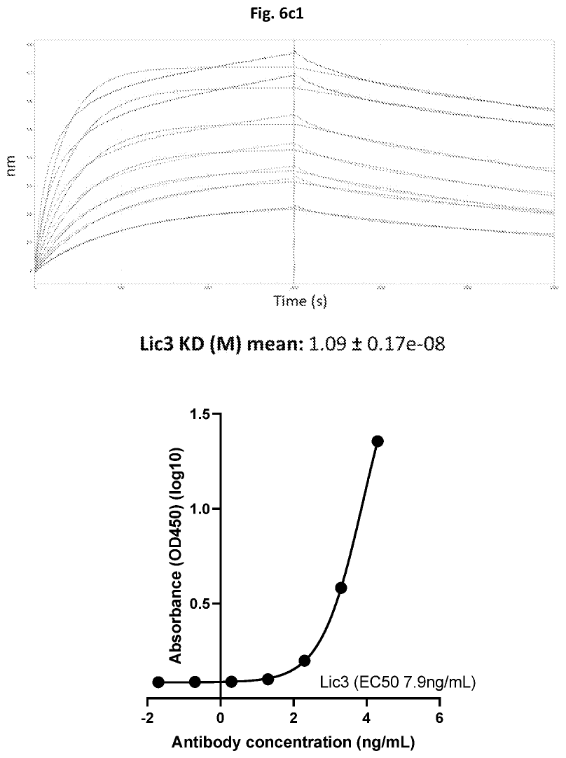

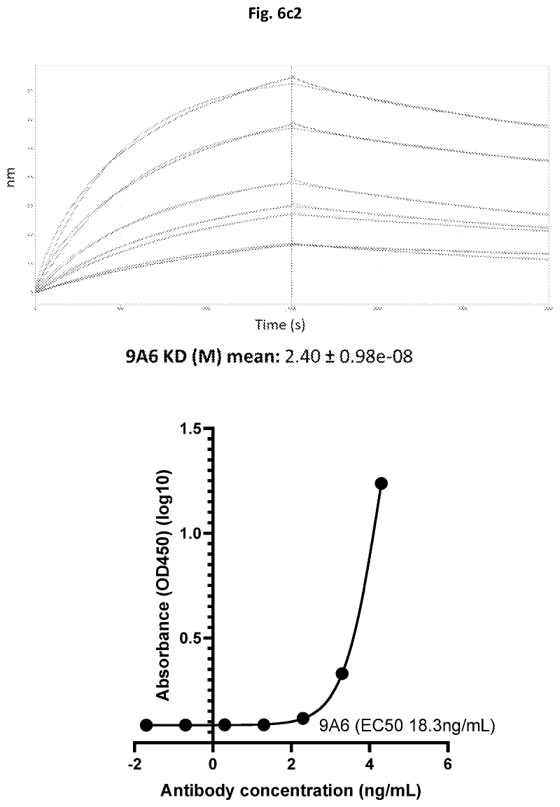

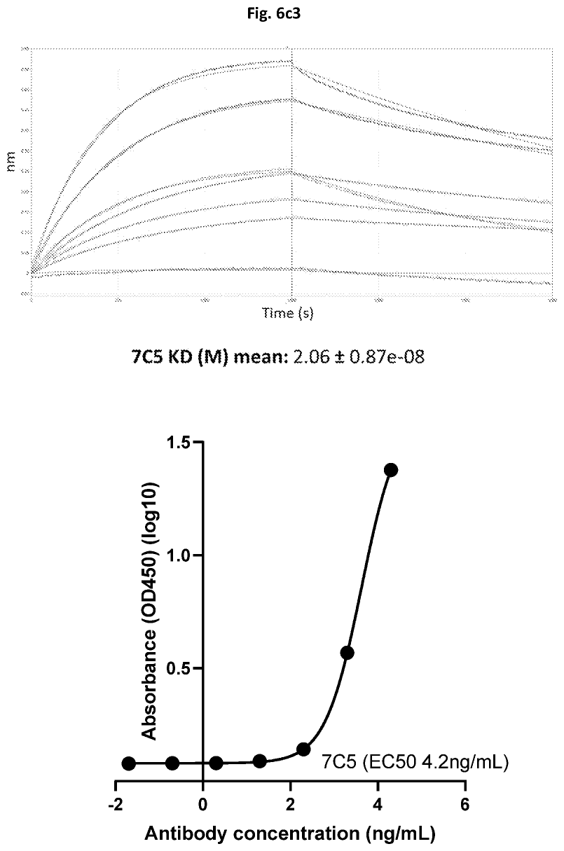

Capture antibodies: In one embodiment, the assay shown herein is useful for the clinical application of CDH17 for CRC diagnostics. Considering that circulating CDH17 may exist in multiple forms—from freely circulating total protein, truncated protein, exosome associated protein—a high emphasis was laid on the identification of potential antibodies that can assist in the capture of the majority forms of CDH17 from the liquid biopsy. Using a panel of over 400 CDH17 monoclonal antibodies (see PCT/US2019/032752), several antibodies have been screened and identified. These antibodies can be potentially used as a capture or detection antibodies for CDH17 detection. To localize each epitope recognized by each of these antibodies, the domain mapping was carried out using truncated human CDH-17 peptides in an ELISA assay (FIG. 6b). Moreover, the target specificity for each of these antibodies towards CDH17 was assessed using polyacrylamide gel electrophoresis followed by Coomassie staining (FIG. 6a). Finally, for the shortlisted capture antibodies such as 7C5, Lic3, and 9A6 (SEQ ID NO. 1-6), target affinity was determined using ELISA and OCTET (FIG. 6c).

Detection antibodies: The detection antibodies were selected based on their high affinity towards CDH17 and its property of binding to one or ectodomains of CDH17, which were different from the capture antibody. Using purified CDH17-hFc as a standard, the sensitivity of the assay was determined using CDH17-BLI assay, CDH17-ELISA, and CDH17-TRFIA and it was found to extend down to pg/mL for all the assays (FIG. 5b).

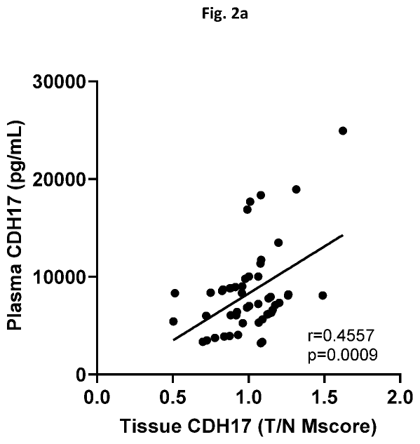

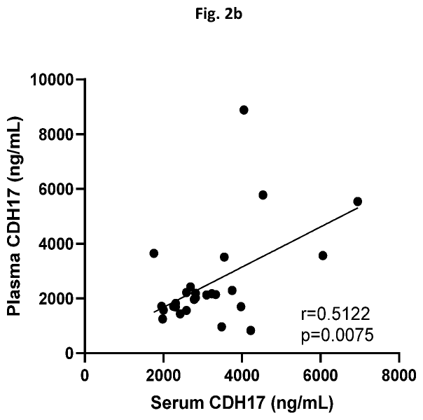

(B) Choice of sample type. While FFPE tissues obtained from cancerous or normal individuals showed a distinct expression pattern of CDH17, an important outcome observed was a high concordance between this expression pattern with the plasma expression of CDH17 for the given individuals (FIG. 2a). An obvious debate between the sample choice also exists with serum or plasma. Within our study pool, serum and plasma was extracted from individuals and CDH17 expression within each sample was analyzed. A high concordance was discovered between the expression of CDH17 within either sample type indicating that both blood sample subtypes serve as an optimal choice for the analyzing the expression of CDH17 (FIG. 2b).

(C) Working with recombinant anti-CDH17 antibodies. To improve the efficacy and the assay flexibility, recombinant capture anti-CDH17 antibodies incorporating at least one Avitag for biotinylation were developed. Moreover, detection antibodies could also incorporate Avitag for biotinylation and conjugation to HRP streptavidin, or a europium-streptavidin conjugate.

### EXAMPLES

### Example 1. Methods for Sample Preparation

Blood specimens from individuals were collected and processed to extract serum and plasma samples. The extracted serum and plasma samples were either assayed immediately or stored at −80° C. until further use. Comparison of the assay performance between the two sample types were performed to identify its influence over the overall CDH17 detection efficiency by the assay (FIG. 2b) indicating a strong concordance of CDH17 expression within the serum and plasma samples. Moreover, the plasma expression of CDH17 showed a strong correlation with the tissue expression of the protein (FIG. 2a).

### Example 2. Methods for Characterizing the Circulating CDH17 Protein

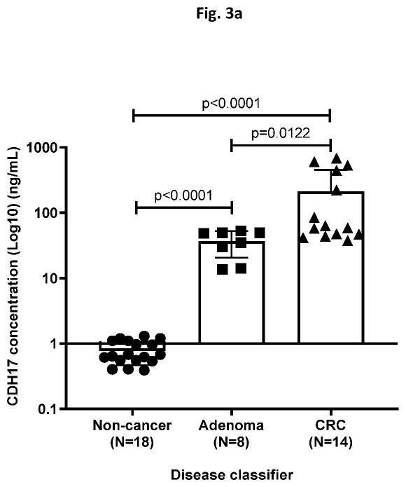

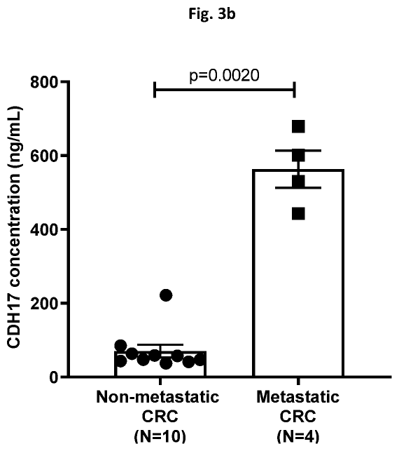

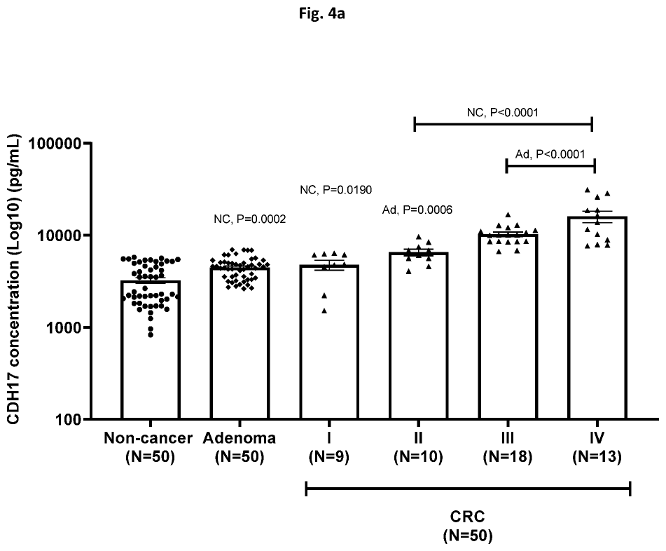

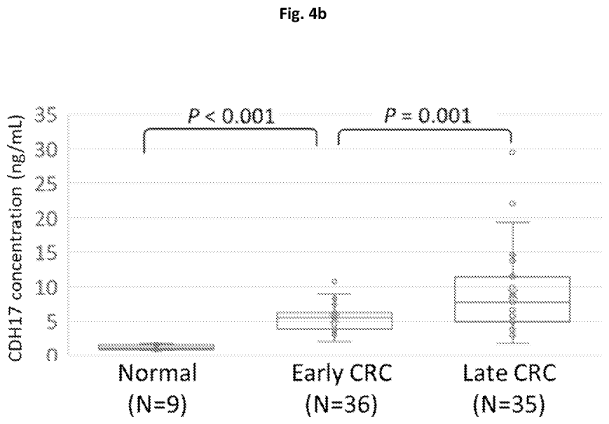

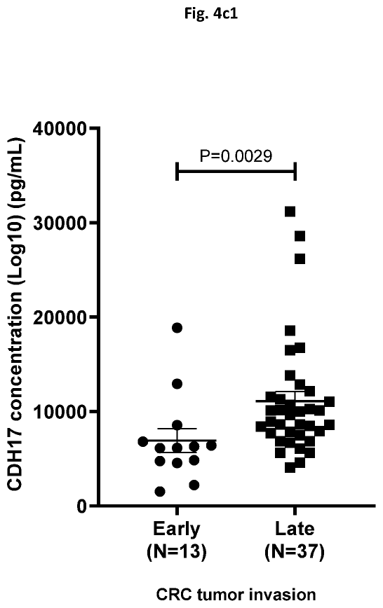

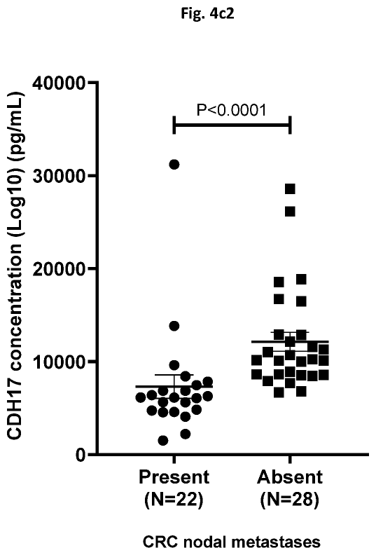

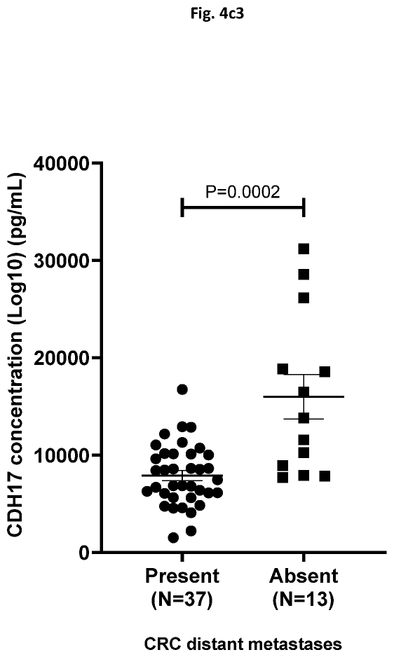

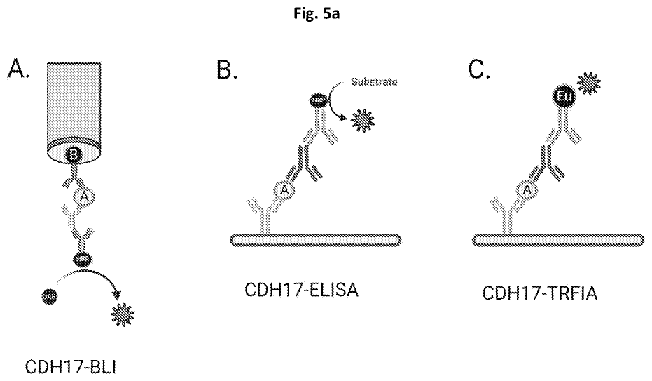

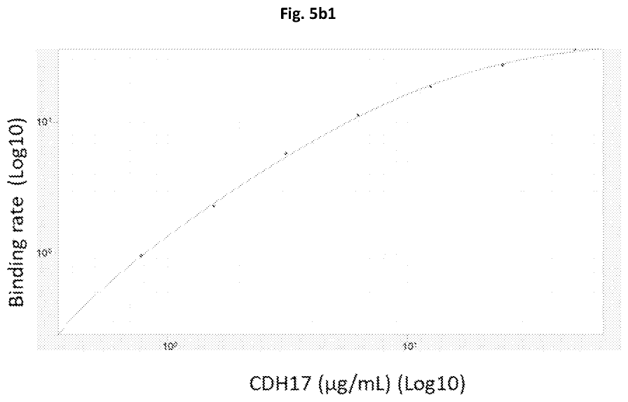

The expression of CDH17 protein within circulation can be quantified using several methods including immunohistochemistry, BLI-assay, ELISA, and TRFIA. Using the methods disclosed herein, CDH17 protein is easily detectable in the gastrointestinal system—with the cancers showing a median to high expression range of almost 1 ug/mL whereas the non-cancer tissues show an extremely low protein expression at sub-nanomolar or negligible range (FIGS. 3 and 4). Additionally, expression of CDH17 was significantly higher in individuals with precancerous adenomatous polyps relative to the non-cancer group (FIGS. 3 and 4). A personal or a family history of colorectal polyps has been identified as a strong risk factor for the development of CRC. Similar pre-cancerous lesions or nodules in other GI cancers which bear a high risk for the development of a malignant disease serve as potential diagnostic timepoints for the early detection of the developing cancer. Early identification of such pre-cancerous lesions, benign polyps or nodules within the GI canal using CDH17 as a diagnostic marker bears a significant clinical relevance in improving overall disease outcomes.

Moreover, the expression of CDH17 within the cancer tissues increased with a relative increase in the overall tumor burden in terms of greater invasion, and the development of nodal and distant metastases. Multiple studies have indicated a direct correlation of CDH17 expression with an advanced disease presentation and an overall poor prognosis in cancers showing a high expression of CDH17 (Wang et al, 2013; Tsoi et al, 2013; Wang et al, 2005; Kaposi-Novak et al, 2006; Lee et al, 2018; Liu et al, 2012). Non-invasive estimation of CDH17 within the blood samples of GI cancer patients can consequently allow the prognostic monitoring of the disease.

CDH17 protein can be released from the developing tumor or the pre-tumor stage in multiple forms—free, conjugated, enveloped, etc. Furthermore, with an extracellular domain length comprising of 6 domains, the protein can also exist in a complete or a truncated form. Consequently, identification of high value capture antibodies that can maximally bind to the circulating protein provide a significant technological advantage of improved detection sensitivity by ensuring the near total concentration of the protein is determined. For development of a clinically applicable diagnostic assay, a panel of over 400 CDH17 monoclonal antibodies has been generated with epitopes mapping to all 7 CDH17 ectodomains (see PCT/US2019/032752). Incorporating multiple capture antibodies within a single assay that can target different domains of the CDH17 protein (FIG. 6) allows for a better visibility (and sensitivity) of the total CDH17 protein concentration as compared to a single antibody-based capture.

Several markers are used routinely in the clinics for the diagnosis and the prognostic monitoring of multiple GI cancers. For CRC, clinical markers such as CEA (carcinoembryonic antigen) and Ca19-9 (Carbonic anhydrase 19-9) are monitored clinically for suspected cases or surveillance for several GI cancers. However, several studies report that these markers suffer from a low diagnostic sensitivity and disease specificity (Gold et al, 1978; Kim et al, 2020; Macdonald, 1999; Eleftheriadis et al, 2009). Compared with the CDH17 diagnostic assay, CEA and Ca19-9 showed a significantly low sensitivity and specificity for the detection of CRC cases (Table 2). This information can be further developed as a prognostic assay for determining the course of tumor development as well as for considering the therapeutic modalities for treating the GI cancer.

### Example 3. The CDH17 Diagnostic Assay Platform

To quantitate the levels of CDH17 in clinical specimens, a standard curve using known dilutions of CDH17 is generated (FIG. 5B). An operational schematic is defined in FIG. 5A for the three assay formats-BLI assay, ELISA, and TRFIA. For the CDH17-BLI assay, the protocol involves the initial immobilization of the biotinylated capture antibodies on the streptavidin probes. Following a subsequent binding to CDH17 standards and detection antibodies, signal is generated using secondary reagents such as metal-enhanced DAB substrate which leads to a sharp jump in the measurable signal of the system. Upon establishment of the standard curve, which is a one-time event, unless the assay protocol is subsequently modified, the CDH17-BLI platform can be directly utilized for analyzing CDH17 expression levels using clinical specimens. For the CDH17-ELISA and CDH17-TRFIA platform, the capture antibodies are immobilized on a hydrophilic microplate surface and are exposed to CDH17 standards or biofluids. For ELISA, the anti-CDH17 secondary antibody may be directly or indirectly conjugated to a peroxidase enzyme which can cause the oxidation of the substrate, and quantification of the amount of the oxidized substrate assists in quantifying the amount of CDH17 within the sample. In contrast to TRFIA, the anti-CDH17 secondary antibody may be directly or indirectly conjugated to europium or biotin (which may be subsequently bound by europium streptavidin), which can then be used to measure the fluorescence shift. This allows the quantification of the amount of CDH17 within the given sample.

While these diagnostic platforms have been applied for liquid biopsies (plasma and serum), these technologies can also be utilized for quantitative estimation of CDH17 levels using other samples including cell culture supernatant, as well as other biofluids such as peripheral blood, serum, plasma, urine, saliva, bone marrow, pleural or peritoneal fluid, or intestinal fluid. The samples can be applied either in an undiluted format or can be diluted in the assay buffer and used for experimental evaluation. The dilution format can be determined based on a preliminary understanding of the basal CDH17 concentration within the sample type—for example non-cancerous specimens have a sub nanomolar concentration of CDH17 while the cancerous specimens have a high probable CDH17 concentration. Following the assay run, the concentration of CDH17 within the samples can be extrapolated using the CDH17 standard curve performed using a standard regression curve.

### Example 4. Clinical Application of the CDH17 Diagnostic Assay

The CDH17 diagnostic assay is a method for determining the amount of CDH17 protein, comprising: a) exposing a capture antibody immobilized on a biosensor or a hydrophilic microplate surface to a sample containing the CDH17 protein, the capture antibody having a binding affinity for the CDH17 protein; b) allowing the CDH17 protein to bind to the capture antibody to provide a bound CDH17 protein; c) allowing a detection molecule to bind to the bound CDH17 protein, wherein the detection molecule comprises a peroxidase enzyme or a fluorescent labeling reagent like europium conjugated to a secondary antibody having an affinity to the CDH17 protein; d) determining the amount of the CDH17 protein based on the amount of the substrate oxidized by the peroxidase enzyme or the amount of fluorescence signal produced; and e) determining the probability of the subject carrying the CDH17 positive precancerous condition or tumor.

The clinical performance of the assay depends on the effective identification of individuals with disease or at risk of disease from the non-cancerous individuals. Colorectal adenomas or polyps have been identified as a strong risk factor for the development of CRC. Consequently, the identification of adenomas at a high diagnostic sensitivity and specificity significantly increases the diagnostic power of the assay. The expression level of CDH17 increases with the disease staging from colorectal adenoma to early CRC to late CRC with the highest expression observed in individuals with metastatic CRC (FIGS. 2 and 3). The extremely high diagnostic sensitivity and specificity of CDH17 diagnostic assay platform in comparison with the current clinically utilized biomarkers such as carcinoembryonic antigen and carbonic anhydrase 19-9 for CRC diagnosis and monitoring (Table 2) allows the application of this technology for population screening of colorectal as well as other GI cancers which have been observed to show a high expression of CDH17. With an initial identification of potential individuals with pre-cancerous conditions such as adenoma or carcinoma using the CDH17 diagnostic assay, the screened candidates can subsequently be assessed by a significantly more expensive invasive tool of colonoscopy for disease severity confirmation.

The disclosed solution is indeed very valuable both in the clinical and the research sectors since it has remained unexplored although the technology has been existent for a few years now. While CDH-17 is an established diagnostic biomarker in several cancers, there have been limited companies that have explored this biomarker for commercialization purposes. The CDH17-diagnostic assay platform can mature into in vitro diagnostic (IVD) technology that is clinically applicable and can be applied for improved disease management of GI cancers.

The pharmaceutical preparations may be in unit dosage forms. In such form, the preparation may be subdivided into unit doses containing appropriate quantities of the active component. The unit dosage form can be a packaged preparation, such as a kit or other form, the package containing discrete quantities of preparation, such as packeted tablets, capsules, liquids or powders in vials or ampoules. Also, the unit dosage form can be a capsule, tablet, cachet, or lozenge, or it can be the appropriate number of any of these in packaged form.

The present disclosure is further illustrated by the following examples, which should not be construed as limiting in any way. The contents of all cited references throughout this application are hereby expressly incorporated by reference. The practice of the present disclosure will employ, unless otherwise indicated, conventional techniques of pharmacology and pharmaceutics, which are within the skill of the art.

### Table

TABLE 1
Characteristic reagents for assaying circulating CDH17 protein

| CDH17 capture antibody | CDH17 detection antibody |
|---|---|
| First CDH17 Ab, e.g. | Second CDH17 Ab, e.g. |
| m7C5 (SEQ ID No: 1, SEQ | 10C12 (ARB102) (SEQ ID No: 7, SEQ ID |
| ID No: 2) | No: 8) |
| mLic3 (SEQ ID No: 3, | Commercial monoclonal or polyclonal anti- |
| SEQ ID No: 4) | CDH17 (affinity to D1-D6) antibody |
| m9A6 (SEQ ID No: 5, SEQ | Labelled with HRP, biotin, fluorescent dye |
| ID No: 6) | (europium), etc. |
| labelled with or without | or conjugated to a commercial antibody |
| biotin | labelled with HRP, biotin, fluorescent dye, |
| | etc. |

TABLE 2
Diagnostic accuracy of CDH17 relative to clinically
used standards (CEA/CA19-9) for colorectal cancer

| Biomarker under test | Cut-off | | AUC | Sensitivity | Specificity |
|---|---|---|---|---|---|
| CDH17 (BLI assay) | 7.45 | ng/mL | 1.0000 | 100% | 100% |
| CDH17 (TRFIA) | 1.74 | ng/mL | 1.0000 | 100% | 100% |
| CDH17 (ELISA) | 5.5 | ng/mL | 0.9404 | 86% | 96% |
| CEA | 5 | ng/mL | 0.7000 | 50% | 80% |
| CA19-9 | 37 | U/mL | 0.8167 | 66.67% | 70% |

### Sequence Listing

>SEQ ID NO: 1
Amino acid sequences of m7C5 variable heavy chain domain
QVQLQQSGAELARPGASVKLSCKASGYTFTSYGLSWVKQRTGQGLEWIGEIFPRSGNSYYNEKFKGKAALTA
DKSSSTAYMQLSSLTSEDSAVYFCARHYYSSLYYAMDYWGQGTSVTVSS
>SEQ ID NO: 2
Amino acid sequences of m7C5 variable light chain domain
DIQVTQSPASLSASVGESVSITCGTNENLYGALNWYQRKQGKSPQLLIYGATNLADGMSSRFSGSGSGRQYS
LKISSLHPDDVATYYCQNVLSTPRTFGGGTKLEIK
>SEQ ID NO: 3
Amino acid sequences of mLic3 variable heavy chain domain
EVOLVESGGGLVKPGGSLKLSCAASGFSFSDYYMYWVRQAPEKRLEWVASISFDGTYTYYTDRVKGRFTISR
DNAKNNLYLQMSSLKSEDTAMYYCARDRPAWFPYWGQGTLVTVSA
>SEQ ID NO: 4
Amino acid sequences of mLic3 variable light chain domain
DVLMTQIPLSLTVSLGDQASISCRSSQSIVHSNGNTYLEWYLQRPGQSPKLLIYKVSNRFSGVPDRFSGSGSG
TDFTLKISRVEAEDLGVYYCFQGSHVPLTFGAGTKLELK
>SEQ ID NO: 5
Amino acid sequences of m9A6 variable heavy chain domain
EVKLQESGPELVKPGASVTISCKASGYTFTDYYINWVKQRPGQGLEWIGWLFPGSGTTYYNEKFKGKATLTV
AKSSSTAYMLLSSLTSEDSAVYFCARWGFGNYAFAYWGQGTLVTVSA
>SEQ ID NO: 6
Amino acid sequences of m9A6 variable light chain domain
DIVLTQSQKFMSATVGDRVSITCKASQNVGTAVAWYQQKPGQSPKLLIYSPSSRNTGVPDRFTGSGSGTDF
TLTISSVQSEDLADYFCQQYSTYPRTFGGGTKLEIK
>SEQ ID NO: 7
Humanized amino acid sequences of 10C12 variable heavy chain domain (ARB102)
EVOLVESGGGLVQPGGSLRLSCAASGFTFSSYAMSWVRQTPGKGLEWVAVIDSNGGSTYYPDTVKDRFTIS
RDNSKNTLYLQMNSLRAEDTAVYYCSSYTNLGAYWGQGTLVTVSA
>SEQ ID NO: 8
Humanized amino acid sequences of 10C12 variable light chain domain (ARB102)
DIQMTQSPSSLSASVGDRVTITCRASQDISGYLNWLQQKPGGAIKRLIYTTSTLDSGVPKRFSGSGSGTDFTLT
ISSLQSEDFATYYCLQYASSPFTFGGGTKVEIK
>SEQ ID NO: 9
m7C5 heavy chain CDR-H1 sequence
GYTFTSYG
>SEQ ID NO: 10
m7C5 heavy chain CDR-H2 sequence
IFPRSGNS
>SEQ ID NO: 11
m7C5 heavy chain CDR-H3 sequence
ARHYYSSLYYAMDY
>SEQ ID NO: 12
m7C5 light chain CDR-L1 sequence
ENLYGA
>SEQ ID NO: 13
m7C5 light chain CDR-L2 sequence
GAT
>SEQ ID NO: 14
m7C5 light chain CDR-L3 sequence
QNVLSTPRT
>SEQ ID NO: 15
mLic3 heavy chain CDR-H1 sequence
DYYMY
>SEQ ID NO: 16
mLic3 heavy chain CDR-H2 sequence
SISFDGTYTYYTDRVKG
>SEQ ID NO: 17
mLic3 heavy chain CDR-H3 sequence
DRPAWFPY
>SEQ ID NO: 18
mLic3 light chain CDR-L1 sequence
RSSQSIVHSNGNTYLE
>SEQ ID NO: 19
mLic3 light chain CDR-L2 sequence
KVSNRFS
>SEQ ID NO: 20
mLic3 light chain CDR-L3 sequence
FQGSHVPLT
>SEQ ID NO: 21
m9A6 heavy chain CDR-H1 sequence
DYYIN
>SEQ ID NO: 22
m9A6 heavy chain CDR-H2 sequence
WLFPGSGTTYYNEKFKG
>SEQ ID NO: 23
m9A6 heavy chain CDR-H3 sequence
WGFGNYAFAY
>SEQ ID NO: 24
m9A6 light chain CDR-L1 sequence
KASQNVGTAVA
>SEQ ID NO: 25
m9A6 light chain CDR-L2 sequence
SPSSRNT
>SEQ ID NO: 26
m9A6 light chain CDR-L3 sequence
QQYSTYPRT
>SEQ ID NO: 27
10C12 heavy chain CDR-H1 (ARB102) sequence
GFTFSSYA
>SEQ ID NO: 28
10C12 heavy chain CDR-H2 (ARB102) sequence
IDSNGGST
>SEQ ID NO: 29
10C12 heavy chain CDR-H3 (ARB102) sequence
SSYTNLGAY
>SEQ ID NO: 30
10C12 light chain CDR-L1 (ARB102) sequence
QDISGY
>SEQ ID NO: 31
10C12 light chain CDR-L2 (ARB102) sequence
TTS
>SEQ ID NO: 32
10C12 light chain CDR-L3 (ARB102) sequence
LQYASSPFT

What is claimed is:

1. A method for screening a subject for cancer by determining the amount of CDH17 protein in a sample from the subject, the method comprising:
contacting the sample to a capture antibody having a binding affinity to CDH17, wherein any CDH17 protein in the sample is configured to bind to the capture antibody to provide a bound sample,
contacting the bound sample to a detection molecule to provide a detection sample, wherein the detection molecule comprises a sensing signal molecule conjugated to a secondary antibody having a binding affinity to the CDH17 protein,
generating a sensing signal through the sensing signal molecule bound to the detection sample,
determining the amount of the sensing signal, and
determining the amount of the CDH17 protein in the sample based on the amount of the sensing signal.

2. The method of claim 1, further comprising determining the probability of the subject carrying CDH17 positive precancerous condition or CDH17 positive cancer based on the amount of CDH17 protein in the sample.

3. The method of claim 1, wherein the sensing signal molecule comprises a peroxidase enzyme and the sensing signal comprises an oxidized substrate, and wherein the step of generating the sensing signal through the sensing signal molecule comprises oxidizing a substrate with the peroxidase enzyme to provide the oxidized substrate.

4. The method of claim 1, wherein the sensing signal molecule comprises a fluorescent labeling reagent and the sensing signal comprises fluorescence signal.

5. The method of claim 1, wherein the capture antibody comprises an amino acid sequence having at least 98% sequence identity to SEQ ID NO: 1, 2, 3, 4, 5, or 6.

6. The method of claim 1, wherein the capture antibody comprises
3 heavy chain complimentary determining regions (CDRs) having the SEQ ID NO: 9, 10, 11 and 3 light chain CDRs having the SEC ID NO: 12, 13, 14,
3 heavy chain CDRs having the SEQ ID NO: 15, 16, 17 and 3 light chain CDRs having the SEQ ID NO: 18, 19, 20, or
3 heavy chain CDRs having the SEQ ID NO: 21, 22, 23 and 3 light chain CDRs having the SEQ ID NO: 24, 25, 26.

7. The method of claim 1, wherein the secondary antibody comprises an amino acid sequence having at least 98% sequence identity to SEQ ID NO: 7-8.

8. The method of claim 1, wherein the secondary antibody comprises 3 heavy chain CDRs having the SEQ ID NO: 27, 28, 29 and 3 light chain CDRs having the SEC ID NO: 30, 31, 32.

9. The method of claim 1, wherein the capture antibody is carried on a biosensor.

10. The method of claim 1, wherein the cancer comprises a CDH17 positive cancer of gastrointestinal system.

11. The method of claim 1, wherein the sample comprises a bodily fluid.

12. The method of claim 11, wherein the bodily fluid comprises peripheral blood, serum, plasma, urine, saliva, bone marrow, pleural or peritoneal fluid, or intestinal fluid.

13. A monoclonal antibody having an affinity to CDH17, comprising,
3 heavy chain CDRs having the SEQ ID NO: 9, 10, 11 and 3 light chain CDRs having the SEQ ID NO: 12, 13, 14,
3 heavy chain CDRs having the SEQ ID NO: 15, 16, 17 and 3 light chain CDRs having the SEQ ID NO: 18, 19, 20.
3 heavy chain CDRs having the SEQ ID NO: 21, 22, 23 and 3 light chain CDRs having the SEQ ID NO: 24, 25, 26, or
3 heavy chain CDRs having the SEQ ID NO: 27, 28, 29 and 3 light chain CDRs having the SEQ ID NO: 30, 31, 32.

14. The monoclonal antibody of claim 13, comprises an amino acid sequence having at least 98% sequence identity to SEQ ID NO 1, 2, 3, 4, 5, 6, 7, or 8.
